(19) United States
(12) Patent Application Publication
(10) Pub. No.: US 2004/0247577 A1
(43) Pub. Date: Dec. 9, 2004

(54) Method and composition for inhibiting heparanase activity

(76) Inventors: Peter Bohlen, New York, NY (US); Daniel J. Hicklin, Upper Montclair, NJ (US); Paul Kussie, New York, NY (US); Yiwen Li, Jersey City, NJ (US)

Correspondence Address: KENYON & KENYON, ONE BROADWAY, NEW YORK, NY, 10004, US

(21) Appl. No.: 10/483,858

(22) PCT Filed: Jul. 10, 2002
(86) PCT No.: PCT/US02/21773

Related U.S. Application Data
(60) Provisional application No. 60/305,446, filed on Jul. 13, 2001.

(51) Int. Cl. A61K 48/00; A61K 39/395

(52) U.S. Cl. 424/93.21; 424/184.1; 424/146.1

(57) ABSTRACT

The present invention relates to methods of inhibiting heparanase activity and treating various conditions by administering to an animal an effective amount of an immunogen that elicits an immune response to heparanase. According to the present invention, the immunogen is heparanase or a fragment thereof and, preferably, the immunogen is an antigen presenting cell (APC), such as a dendritic cell (DC), displaying heparanase or a fragment thereof on the surface. The present invention also relates to compositions containing the immunogen.

### FIELD OF THE INVENTION

The present invention is directed to methods and compositions for inhibiting heparanase activity. More particularly, the present invention is directed to methods for treatment of conditions associated with heparanase activity.

### BACKGROUND OF THE INVENTION

Heparan sulfate proteoglycans (HSPGs) are widely distributed in mammalian tissues and are involved in a number of processes related to malignancy. See generally Blackhall et al., Br. J Cancer, 85(8): 1094-8 (Oct. 2001). Elevated levels of heparanase have been detected in sera from metastatic tumor-bearing animals and malignant melanoma patients, and a correlation exists between serum heparanase activity and the extent of tumor metastases. Cleavage of HSPGs by heparanase leads to disassembly of ECM and release of bioactive agents such as pro-angiogenic factors. The successful penetration of endothelial basement membranes is an important process in the formation of hematogenous tumor metastases. Heparanase-inhibiting, non-anticoagulant species of heparin, as well as laminarin sulfate and mannopentaose phosphate sulfate (PI-88), markedly reduced the incidence of lung metastasis in experimental animals, Vlodavsky et al., 1994, supra; Miao et al., Int. J. Cancer, 83: 424-31 (1999); Nakajima, 1988, supra; Parish et al., Int. J. Cancer, 40: 511-7 (1987), as well as tumor growth and angiogenesis, Parish et al., Cancer Res., 59: 3433-41 (1999), suggesting that heparanase is potentially a useful marker for tumor development. Thus, immunotherapy targeting heparanase activity may be a potentially useful treatment for both tumor growth and angiogenesis.

These non-anticoagulant species of heparin also significantly impaired the traffic of T lymphocytes and suppressed cellular immune reactivity and experimental autoimmune diseases. Vlodavsky et al., 1992, supra. Furthermore, treatment with heparanase inhibitors markedly reduced the incidence of experimental autoimmune encephalomyelitis, adjuvant arthritis and graft rejection, see Vlodavsky et al., 1992, supra; Lider et al., J. Clin. Invest., 83: 752-6 (1989); Willenborg & Parish, J. Immunol., 140: 3401-5 (1988), indicating that immunotherapeutic treatments targeting heparanase activity may be potentially useful for these conditions.

HSPGs are composed of a core protein to which chains of the glycosaminoglycan, heparan sulfate (HS), are attached. The polysaccharide HS chains are typically composed of repeating hexuronic and D-glucosamine disaccharide units that are modified at various positions by sulfation, epimerization and N-acetylation, yielding clusters of sulfated disaccharides separated by low or non-sulfated regions. The existence of various classes of core protein, in addition to highly polymorphic HS chains, creates a superfamily of macromolecules with considerable diversity of structure and function.

HSPGs interact with many proteins, including growth factors, chemokines and structural proteins of the extracellular matrix (ECM), to influence cell growth, differentiation, and the cellular response to the environment. Specifically, interaction of T and B lymphocytes, platelets, granulocytes, macrophages and mast cells with the subendothelial ECM is associated with degradation of HS by a specific, endo-β-D-glucuronidase (heparanase) activity. See Nakajima et al., Science, 220: 611-613 (1983). Studies have also shown that HSPGs play an important role in the self-assembly and insolubility of ECM components, as well as in cell adhesion and locomotion. See Bermfield et al., Annu. Rev. Biochem., 68: 729-77 (1999); Iozzo, Annu. Rev. Biochem., 67: 609-52 (1998); Vlodavsk et al., Invasion & Metastasis, 14: 290-302 (1994); Wight et al., Curr. Opin. Cell Biol., 4: 793-801 (1992).

The heparanase enzyme that degrades HS is released from intracellular compartments, for example, from lysosomes and specific granules, in response to various activation signals, such as thrombin, calcium ionophore, immune complexes, antigens and mitogens, suggesting its regulated involvement in inflammation and cellular immunity. Heparanase expressed by intact cells, platelets, mast cells, neutrophils and lymphoma cells was found to release active HS-bound basic fibroblast growth factor (bFGF) from ECM and basement membranes. Heparanase can thus elicit an indirect neovascular response in processes such as wound repair (resulting from injury) and inflammation. See generally Vlodavsky et al., Invasion Metastasis, 12(2): 112-27 (1992); Nakajima et al., J. Cell Biochem., 36(2): 157-67 (1988).

The genes for human heparanase (see Vlodavsky et al., Nat. Med., 5: 793-802 (1999); Hulett et al., Nat. Med., 5: 803-809 (1999); Kussie et al., Biochem. Biophy. Res. Commun., 261: 183-187 (1999)) and chicken heparanase (see Toyoshima & Nakajima, J. Biol. Chem., 261: 183-187 (1999)) have been cloned and functionally expressed. The complete sequence of bovine heparanase (gene bank: AF281160), rat heparanase (gene bank: NM₁₃ 022605) and a partial sequence (residues 150-535) of murine heparanase (gene bank: AX034647) (see Hulett et al., supra) are known. In addition, GenBank accession number AX034647 (see EP 1032656-A) discloses another partial sequence (residues 156-535), along with the corresponding polynucleotide sequence, of murine heparanase.

### SUMMARY OF THE INVENTION

The present invention is directed to methods of inhibiting heparanase activity and treating various conditions by administering to an animal an effective amount of an immunogen that elicits an immune response to heparanase. The present invention provides methods whereby the immunogen is heparanase or a fragment thereof and, in a preferred embodiment, the immunogen is an antigen presenting cell (APC), such as a dendritic cell (DC), displaying heparanase or a fragment thereof on the surface. Also provided by the present invention are compositions of the immunogen.

Additionally, the present invention is directed to an isolated heparanase mouse polypeptide, for example, SEQ ID NO:1 or a fragment thereof. In addition, the present invention is directed to an isolated heparanase polynucleotide encoding a mouse heparanase polynucleotide, for example, SEQ ID NO:2 or a fragment thereof, and a cloning (or expression) vector and a host cell having such a polynucleotide.

### BRIEF DESCRIPTION OF THE DRAWINGS

FIG. 1 graphically depicts the concentration of heparanase specific antibodies at various dilutions induced in an animal as a result of in vivo administration to the animal of a control polypeptide.

FIG. 2 graphically depicts the concentration of heparanase specific antibodies at various dilutions induced in an animal as a result of in vivo administration to the animal of DC pulsed with a control polypeptide.

FIG. 3 graphically depicts the concentration of heparanase specific antibodies at various dilutions induced in an animal as a result of in vivo administration to the animal of DC pulsed with heparanase.

FIG. 4 graphically depicts the number of Elispots producing INF-γ in an animal at different dilutions as a result of in vivo administration to the animal of a control polypeptide.

FIG. 5 graphically depicts the number of Elispots producing INF-γ in an animal at different dilutions as a result of in vivo administration to the animal of DC pulsed with a control polypeptide.

FIG. 6 graphically depicts the number of Elispots producing IFN-γ in an animal at different dilutions as a result of in vivo administration to the animal of DC pulsed with heparanase.

FIG. 7 graphically depicts the percent survival over time of an animal following in vivo administration to the animal of PBS, DC pulsed with a control polypeptide, or DC pulsed with heparanase.

FIG. 8 graphically depicts the mean number of lung metastases in an animal following in vivo administration to the animal of PBS, DC pulsed with a control polypeptide, or DC pulsed with heparanase.

### DETAILED DESCRIPTION OF THE INVENTION

In the context of the present invention, heparanase, heparanase activity or heparanase catalytic activity refers to an animal endoglycosidase hydrolyzing activity that is specific for heparan or HS (including HSPG) substrates. This is in contrast to the activity of bacterial enzymes (heparinase I, II and III) that degrade heparan or HS by means of β-elimination. Heparanase activity that is inhibited in the context of the present invention is preferably native. That is, the heparanase activity, while it may be altered relative to basal levels, is naturally occurring in the animal.

In one embodiment, the present invention is directed to inhibiting heparanase activity by eliciting an immune response to heparanase. One consequence of inhibition of heparanase activity is prevention of degradation of HSPG. It should be appreciated that this is but one consequence and is by no means the only consequence of inhibition of heparanase activity. Other consequences include inhibition of degradation of the basement membrane, which prevents tumor metastasis, and prevention of invasion by endothelial cells, which is involved in angiogenesis. Furthermore, inhibition of heparanase activity may prevent activated cells of the immune system from entering the circulatory system, thus inhibiting elicitation of both inflammatory-related conditions and autoimmune-related conditions.

Heparanase activity is inhibited by elicitation of an immune response to heparanase following administration of an effective amount of one or more immunogen(s). In the context of the present invention, an immunogen is effective to elicit an immune response, including a humoral or cell-mediated immune response, against native heparanase. The immune response is preferably an active immunity that inhibits, that is, prevents, slows, or stops, heparanase activity. Therefore, in the context of the present inventive methods, heparanase activity need not be completely abrogated. It should be appreciated that the immune response against heparanase can be elicited either directly or indirectly. Thus, an immunogen is not required by the present invention to inhibit heparanase activity directly. Rather, the immunogen can elicit the immune response indirectly by initiating a cascade through which heparanase activity is ultimately inhibited. Examples of such indirect inhibition include, but are not limited to, altering the rate or extent of transcription or degradation of one or more species of RNA relating to heparanase activity, translation or post-translational processing of the heparanase polypeptide and/or heparanase protein degradation.

The immunogen of the present invention can be a peptide, a DNA, an RNA, a small molecule, or any other suitable immunogenic molecule that inhibits heparanase activity. The immunogen can be native to the animal; however, such an immunogen must be modified to provoke an immune response. The term “native” as used herein means autologous or homologous to an animal, such that native antigens are “self” polypeptides and are, absent modification, typically non-immunogenic in the animal from which they are derived. Alternatively, the immunogen can be non-native to the animal, meaning foreign and not a “self” polypeptide. As such, these immunogens can induce an immune response without additional modification.

In one embodiment of the present invention, the immunogen is a peptide or polypeptide. Any suitable peptide or polypeptide can be used that inhibits heparanase activity, an example of which is heparanase or a fragment thereof. See U.S. Pat. No. 5,968,822. A suitable heparanase may be a synthetically derived heparanase or a fragment thereof, a recombinantly derived heparanase or a fragment thereof, or a naturally derived heparanase or a fragment thereof. Furthermore, a suitable heparanase can be native or foreign to the animal.

For example, a suitable polypeptide immunogen is human heparanase, which is a 61.2 kDa polypeptide of 543 amino acids. The mature 50 kDa enzyme, isolated from cells and tissues, has it N-terminus 157 amino acids downstream from the initiation codon, suggesting post-translational processing of the heparanase peptide. See, e.g., Kussie et al., Biochem. Biophys. Res. Commun. 261: 183-7 (1999); Toyoshima & Nakajima, J. Biol. Chem. 274: 24153-60 (1999). The amino acid sequence of heparanase contains a putative N-terminal signal peptide sequence (Met¹ to Ala³⁵) and a candidate transmembrane region (Pro⁵¹⁵ to Ile⁵³⁴). See, e.g., Vlodavsky et al., Nat. Med. 5: 793-802 (1999); Hulett et al., Nat. Med. 5: 183-7 (1999). Site directed mutagenesis revealed that similar to other TIM-barrel glycosyl hydrolyses, heparanase has a common catalytic mechanism that involves two conserved acidic residues, a putative proton donor at Glu²²⁵ and a nucleophile at Glu³⁴³. See Hulett et al., Biochem. 39: 15659-67 (2000). Alignment of the partial amino acid sequences of human, mouse and rat heparanase corresponding to the 50 kDa human mature enzyme (Lys¹⁵⁸ to Ile ⁵⁴³), revealed 80% to 93% identity. See Hulett et al., 1999, supra.

A suitable foreign heparanase in the context of the present invention can be any heparanase that is non-native to the animal and that can induce an immune response without additional modification. For example, when the animal is a human, the heparanase can be derived from a mammal, such as a rabbit, rat, or mouse. Preferably, when the animal is a human, the heparanase or a fragment thereof is a mouse heparanase or a fragment thereof or a heparanase derived from a mouse heparanase or a fragment thereof.

A suitable polypeptide sequence for a mouse heparanase has been isolated and is set forth in SEQ ID NO: 1. Purification and polypeptide characterization of the mouse heparanase revealed a non-covalent heterodimer consisting of a 43-kDa polypeptide and a 7-kDa peptide, both of which are derived from a single precursor polypeptide. The enzymatic activity of the mouse heparanase was confirmed by its ability to degrade HSPG and inhibition with known heparanase inhibitors. Analysis of this full-length murine heparanase amino acid sequence also revealed approximately a 76% identity when compared with human heparanase.

The mouse heparanase polypeptides of the present invention have been isolated and/or purified. As used herein, “isolated” or “purified” means that a molecule, for example, the polypeptide, is separated from cellular material or other components that naturally accompany it. Typically, for a polynucleotide or polypeptide, it is substantially pure when it is at least 60% (by weight) free from the proteins and other naturally occurring organic molecules with which it is naturally associated. Preferably, the purity of the preparation is at least 75%, more preferably at least 90%, and most preferably at least 99% (by weight) free. A substantially pure polynucleotide or polypeptide can be obtained, for example, by extraction from a natural source, expression of a recombinant nucleic acid encoding the polypeptide, or chemical synthesis. Purity can be measured by any appropriate method, for example, column chromatography, polyacrylamide gel electrophoresis, or HPLC analysis. A chemically synthesized polynucleotide or polypeptide or a recombinant polynucleotide or polypeptide produced in a cell type other than the cell type in which it naturally occurs is, by definition, substantially free from components that naturally accompany it. Accordingly, substantially pure polynucleotides or polypeptides include those having sequences derived from eukaryotic organisms that are produced in E. coli or other prokaryotes. Moreover, it should be appreciated that the term isolated or purified does not refer to a library-type preparation containing a myriad of other sequence fragments.

Equivalents of heparanase or a fragment thereof, including a foreign heparanase or fragment thereof, can also be used in the present invention. Such equivalents include functional equivalents or derivatives, homologues, analogues (or fragments thereof), or mutant forms of the polynucleotide sequence that induce an immune response comparable to that of the heparanase polypeptide. By functional equivalent, the term refers to alterations in the amino acid sequence, including additions, deletions, and substitutions, that do not substantially alter polypeptide characteristics, e.g., charge, IEF, affinity, avidity, conformation, solubility, and retain the specific function or immunological cross-reactivity of the polypeptide. The term functional equivalents includes conservative amino acid substitutions, which involves a change in the amino acid sequence by way of substituting amino acids of the polypeptide with amino acids having generally similar properties, e.g., acidic, basic, aromatic, size, positively or negatively charged, polarity, non-polarity. The term bomologue refers to a polypeptide sequence from a different species having equivalent characteristics and/or function. Mutant forms refer to alterations of the polypeptide sequence, arising due to splicing, polymorphisms, or other events and which may have been selected naturally.

In addition, such equivalents are immunologically cross-reactive with their corresponding polypeptide; although it should be noted that there can be peptides that inhibit heparanase activity and function as an immunogen in the context of the present invention that do not have similar biological activity to that of native heparanase. The equivalent can also be a fragment of the polypeptide, or a substitution, addition or deletion mutant of the polypeptide, for example. Equivalent polypeptides have equivalent amino acid sequences. An amino acid sequence that is substantially the same as another sequence, but that differs from the other sequence by one or more substitutions, additions and/or deletions, is considered to be an equivalent sequence. Preferably, less than 25%, more preferably less than 10% and most preferably less than 5% of the number of amino acid residues in a sequence are substituted for, added to or deleted from the polypeptides of the invention.

A heparanase fragment of the present invention preferably contains sufficient amino acid residues to define an epitope of the antigen. The fragment can be, for example, a minigene encoding only the epitope. Methods for isolating and identifying immunogenic fragments from known immunogenic polypeptides are described, for example, by Salfeld et al. in J. Virol., 63: 798-808 (1989) and by Isola et al. in J. Virol., 63: 2325-34 (1989). If the fragment defines a suitable epitope, but is too short to be immunogenic, it can be conjugated to a carrier molecule. Some suitable carrier molecules include keyhole limpet hemocyanin, Ig sequences, TrpE and human or bovine serum albumin. Conjugation can be carried out by methods known in the art (described in more detail below).

Preferably, the immunogen of the present invention is an APC containing heparanase or a fragment thereof, which is displayed on the surface of the APC. APCs are generally eukaryotic cells with major histocompatibility complex (MHC), either class I or class II, gene products at their cell surface. Some examples of APCs that can be used in the present invention include DC, as well as macrophages, preferably MHC class II positive macrophages, monocytes, preferably MHC class II positive monocytes, and lymphocytes. See generally U.S. Pat. No. 5,597,563.

Preferably, the APC of the present invention is a DC, which are widely considered the most potent APC and an efficient initiator of immune responses in vivo, including CD4⁺ T helper, CD8⁺ CTL and antibody responses. Also, DCs express high levels of MHC class II molecules and costimulatory molecules important for antigen presentation, such as CD80, CD86, CD40 ligand and ICAM-1. Inoculation of mice with small numbers of DC pulsed with peptide or polypeptide, whole protein or transfected with DNA or RNA has been shown to induce strong T cell-mediated responses in vivo and to elicit a strong immune response, overcoming tolerance to self-antigens.

Any suitable method can be used to introduce the heparanase or a fragment thereof into the APC. One suitable method is to pulse the APC with the heparanase or a fragment thereof. Another suitable method is to introduce a DNA or RNA encoding heparanase or a fragment thereof into the APC, which is then transcribed and/or translated into the heparanase or a fragment thereof within the APC. This DNA or RNA can be introduced into the APC by any suitable method, such as through calcium phosphate transfection or insertion via a cloning or expression vector containing the DNA or RNA encoding heparanase or a fragment thereof, which is described in further detail below.

Although either a full-length heparanase or a fragment thereof can be introduced into the APC, in the context of the present invention, there are several advantages when a full-length heparanase is used. First, due to intracellular processing of the antigen by the APC, multiple CTL epitopes can be expressed. In addition, multiple T helper epitopes can also exist within the entire heparanase polypeptide and such MHC class II determinants can be useful in establishing a sustained cellular response to the antigen. Finally, B cell epitopes can also be present and lead to induction of a heparanase-specific antibody response.

In an alternative embodiment of the present invention, the APC can be a bacterial cell or a eukaryotic cell, such as a peripheral blood cell, that expresses exogenous DNA or RNA encoding the heparanase or a fragment thereof. An example of a suitable bacterial cell is an avirulent strain of Mycobacterium bovis, such as bacille Calmette-Guerin (BCG), or an avirulent strain of Salmonella, such as S. typhimurium. The bacterial cells can be prepared by cloning DNA having the active portion of the antigen (e.g., heparanase) in an avirulent strain, as is known in the art, see, e.g., Curtiss et al., Vaccine, 6: 155-60 (1988) and Galan et al., Gene, 94: 29-35 (1990) for preparing recombinant Salmonella and Stover et al., Vaccines, 91: 393-8 (1991) for preparing recombinant BCG.

The present invention also provides anti-idiotypic antibodies or fragments thereof that mimic heparanase molecules. Anti-idiotypic antibodies are directed against the antigen specific part of the sequence of an antibody or T-cell receptor and thus recognize the binding sites of other antibodies. In principle, an anti-idiotype antibody should inhibit a specific immune response and they are important to the regulation of the immune system. Anti-idiotypic heparanase-specific antibodies can be obtained by methods known in the art. See generally Jerne et al., EMBO 1: 234 (1982); Jerne, Ann. Immunol. (Paris) 125C: 373 (1974).

Alternatively, peptidomimetics (peptide mimetics) can function as immunogens in the context of the present invention. A peptide mimetic is a molecule that mimics the biological activity of a peptide, yet is no longer peptidic in chemical nature. By strict definition, a peptidomimetic is a molecule that no longer contains any peptide bonds, i.e., amide bonds between amino acids; however, in the context of the present invention, the term peptide mimetic is intended to include molecules that are no longer completely peptidic in nature, such as pseudo-peptides, semi-peptides and peptoids. Whether completely or partially non-peptide, peptidomimetics according to this invention provide a spatial arrangement of reactive chemical moieties that closely resembles the three-dimensional arrangement of active groups in the peptide on which the peptidomimetic is based. The techniques of developing peptidomimetics are conventional. Thus, non-peptide bonds that allow the peptidomimetic to adopt a similar structure to the original peptide can replace peptide bonds. Replacing chemical groups of the amino acids with other chemical groups of similar structure can also be used to develop peptidomimetics.

In another embodiment, the immunogen can be a DNA or RNA encoding the heparanase or a fragment thereof. Any DNA or RNA encoding a heparanase or a fragment thereof that elicits an immune response to heparanase and thereby inhibiting heparanase activity is suitable for use in the context of the present invention. For example, a suitable DNA encodes the human heparanase, which contains an open reading frame of 1629 bp. See, e.g., Kussie et al., 1999, supra; Toyoshima & Nakajima, 1999, supra. In addition, a suitable DNA can be a plasmid having the DNA encoding heparanase or a fragment thereof. See, e.g., U.S. Pat. Nos. 5,589,466 and 5,630,796.

Moreover, a suitable DNA or RNA can encode a heparanase that is native or foreign to the animal. A suitable DNA or RNA encoding a foreign heparanase can be any DNA or RNA that encodes a heparanase that is non-native to the animal and that can induce an immune response without additional modification. For example, when the animal is a human, the DNA or RNA can encode a heparanase that is derived from a mammal, such as a rabbit, rat, or mouse. Preferably, when the animal is a human, the DNA or RNA encoding heparanase or a fragment thereof is a DNA or RNA encoding a mouse heparanase or a fragment thereof. A suitable mouse heparanase polynucleotide (DNA), which was cloned from a mouse embryo cDNA library, has been identified; the isolated cDNA sequence encoding it is set forth in SEQ ID NO:2.

Equivalents of DNA or RNA encoding heparanase or a fragment thereof, including DNA or RNA encoding a foreign heparanase, can also be used in the present invention. Such equivalents include DNA, RNA, DNA/RNA duplexes, polypeptide-nucleic acid (PNA), or derivatives thereof that encode functional derivatives or analogues (or fragments thereof) that induce an immune response comparable to that of the heparanase polypeptide. The equivalent can be a fragment of the DNA or RNA, or a substitution, addition or deletion mutant of the DNA or RNA, for example. Equivalent DNAs or RNAs have substantially equivalent nucleic acid sequences. A nucleic acid sequence that is substantially the same as another sequence, but that differs from the other sequence by one or more substitutions, additions and/or deletions, is considered to be an equivalent sequence. Preferably, less than 25%, more preferably less than 10% and most preferably less than 5% of the number of nucleic acid residues in a sequence are substituted for, added to or deleted from the DNA or RNA of the invention.

Further included in the DNA or RNA encoding heparanase or a fragment thereof are degenerate variants, homologues, or mutant forms of the polynucleotide sequence. By degenerate variant, the term refers to changes in polynucleotide sequences, particularly in the third base of the codon, that do not affect the amino acid sequence encoded by the nucleotide sequences. The term homologue refers to a polynucleotide sequence from a different species having equivalent structure and/or function. Mutant forms refer to alterations of the polynucleotide sequence, such as addition, deletion, or substitution of one or more nucleotides using recombinant DNA techniques well known in the art, or which have been selected naturally. Kunkel et al. (1987) Meth. Enzymol. 154: 367-382-382.

The DNA or RNA polynucleotide sequence encoding heparanase or a fragment thereof of the present invention includes fragments or segments that are long enough to use in polymerase chain reaction (PCR) or various hybridization techniques well known in the art for identification, cloning and amplification of all or part of mRNA or DNA molecules. For example, hybridization under high stringency conditions means the following nucleic acid hybridization and wash conditions: hybridization at 42° C. in the presence of 50% formamide; a first wash at 65° C. with 2×SSC containing 1% SDS; followed by a second wash at 65° C. with 0.1×SSC. In addition, the polynucleotides of the present invention include complements of any of the nucleotide or peptides recited above, e.g., cDNA and mRNA.

In addition, the DNA or RNA encoding heparanase or a fragment thereof of the present invention can be introduced into mammalian cells, particularly endothelial cells, by methods known in the art. Such methods have been described, for example, in U.S. Pat. No. 5,674,722. Suitable methods include calcium phosphate transfection or insertion via a cloning or expression vector containing the DNA or RNA encoding heparanase or a fragment thereof. Cells (both eukaryotic and prokaryotic) useful in the context of the present invention can be prepared in vivo or in vitro. Methods for the production of these cells are well known in the art. Suitable cloning or expression vectors for inserting DNA or RNA into eukaryotic cells include well-known derivatives of SV-40, adenovirus, cytomegalovirus (CMV) and retrovirus-derived DNA or RNA sequences. Any such vectors, when coupled with vectors derived from a combination of plasmids and phage DNA (shuttle vectors) allow for the cloning and/or expression of protein coding sequences in both prokaryotic and eukaryotic cells. Other eukaryotic expression vectors are known in the art, see, e.g., Southern & Berg, J. Mol. Appl. Genet., 1: 327-41 (1982); Subramani et al., Mol. Cell. Biol., 11: 854-64 (1981); Kaufrnann & Sharp J. Mol. Biol., 159: 601-21 (1982); Scahill et al., Proc. Natl. Acad. Sci. USA, 80: 4654-9 (1983); Urlaub & Chasin, Proc. Natl. Acad. Sci. USA, 77: 4216-20 (1980).

Some suitable prokaryotic cloning vectors include plasmids from E. coli, such as colE1, pCR1, pBR322, pMB9, pUC, pKSM and RP4. Prokaryotic vectors also include derivatives of phage DNA, such as M13, fd and other filamentous single-stranded DNA phages. For example, vectors for expressing polypeptides in bacteria, especially E. coli, are also known. Such vectors include the pK233 (or any of the tac family of plasmids), T7 and lambda P_{L}. Examples of vectors that express fusion polypeptides are PATH vectors described by Dieckmann and Tzagoloff in J. Biol. Chem., 260: 1513-1520 (1985). These vectors contain DNA sequences that encode anthranilate synthetase (TrpE) followed by a polylinker at the carboxy terminus. Other expression vector systems are based on beta-galactosidase (pEX); lambda P_{L}; maltose binding protein (pMAL); glutathione S-transferase (pGST). See, e.g., Smith & Johnson, Gene, 67: 31-40 (1988); Abath & Simpson, Peptide Research, 3: 167-68 (1990).

Cloning vectors can have segments of chromosomal, non-chromosomal and synthetic DNA sequences. The vectors useful in the present invention contain at least one expression control sequence that is operatively linked to the DNA or RNA sequence or fragment to be expressed, i.e., the DNA or RNA encoding heparanase or a fragment thereof. The control sequence is inserted in the vector in order to control and to regulate the expression of the cloned DNA or RNA sequence. Examples of useful expression control sequences are the lac system, the trp system, the tac system, the trc system, major operator and promoter regions of phage lambda, the control region of fd coat protein and promoters derived from polyoma, adenovirus, retrovirus and simian virus, for example, the early and late promoters of SV40 and other sequences known to control the expression of genes in prokaryotic or eukaryotic cells and their viruses or combinations thereof.

Alternatively, the immunogen of the present invention can be a small molecule. Small molecules of the present invention are entities having carbon and hydrogen atoms, as well as heteroatoms, which include, but are not limited to, nitrogen, sulfur, oxygen, and phosphorus. Atoms in a small molecule are linked together via covalent and ionic bonds; the former is typical for small organic compounds, for example, small molecule tyrosine kinase inhibitors and the latter is typical of small inorganic compounds. The arrangement of atoms in a small organic molecule may represent a chain, for example, a carbon-carbon chain or carbon-heteroatom chain, or ring containing carbon atoms, for example, benzene, or a combination of carbon and heteroatoms, i.e., heterocycles, for example, a pyrimidine or quinazoline. A combination of one or more chains in a small organic molecule attached to a ring system constitutes a substituted ring system and fusion of two rings constitutes a fused policyclic system, which can be referred to as simply a policyclic system. Small molecules include both compounds found in nature, such as hormones, neurotransmitters, nucleotides, amino acids, sugars, lipids and their derivatives, and those compounds made synthetically, either by traditional organic synthesis, bio-mediated synthesis, or a combination thereof. See, e.g., Ganesan, Drug Discov. Today, 7(1): 47-55 (2002); Lou, Drug Discov. Today, 6(24): 1288-1294 (2001). Any suitable small molecule that inhibits heparanase can be used in the context of the present invention, including lipids and polymers of polysaccharides, as well as derivatives thereof, such as, for example, lipopolysaccharides.

In an alternative embodiment, the immunogen of the present invention can be modified in various ways known to one of skill in the art, for example, by co-administering with or conjugating or genetically fusing it to an immunogenic reagent. Conjugation or fusion to an immunogenic reagent can stimulate an immune response or augment the existing immune response elicited by the immunogen. These conjugates and fused molecules can be prepared by any of the known methods for coupling or fusing antigens to carriers or fusion molecules. The conjugates can also be prepared recombinantly as fusion polypeptides by methods well known in the art. The preferred method of conjugation is covalent coupling, whereby the antigen is bound directly to the immunogenic reagent. Moreover, co-administration can be such that the immunogenic reagent is administered prior to, concurrently with, or subsequent to the immunogen.

Preferred immunogenic reagents include polysaccharides, see generally U.S. Pat. No. 5,623,057, and peptidoglycans, see generally U.S. Pat. No. 5,153,173. Other immunogenic reagents include, for example, cytokines, lymphokines, hormones or growth factors. Examples of such molecules include, but are not limited to, chemokines, interferons, granulocyte colony stimulating factor (G-CSF), granulocyte macrophage colony-stimulating factor (GM-CSF), vascular endothelial growth factor (VEGF), stem cell factor (SCF), bFGF and interleukins (IL), such as IL-1, IL-2, IL-3, IL-4, IL-5, IL-6 and IL-7. See generally U.S. Pat. No. 5,334,379. Moreover, the immunogenic reagent can be any suitable medication or therapy conventionally used to prophylactically and/or therapeutically treatment any of the various conditions described herein. For example, when treating a tumor with the present inventive methods, the immunogenic reagent can be a chemotherapeutic agent, radiation, or a receptor antagonist. The immunogen can also be modified by co-administration with or binding to MHC antigen, such as class I and class II restricted antigens, so as to form a complex with the MHC. The source of such MHC antigens and the methods of binding the immunogen to the MHC antigens are described in general in U.S. Pat. No. 4,478,823.

Other suitable methods can be used to modify the immunogen in the context of the present invention. For example, the immunogen can be modified by haptenization (chemically linking) of the immunogen. See generally U.S. Pat. Nos. 4,778,752 and 5,290,551. A hapten is a substance having the ability to, when coupled with a polypeptide, elicit an immune response. The immunogen of the invention can itself be haptenized, or can be bound to hapten-modified proteins. See generally U.S. Pat. Nos. 4,778,752 and 5,290,551. An additional method of modifying the immunogen of the present invention is glycosylation or pegylation of the heparanase or a fragment thereof or glycosylation or pegylation of the carrier molecules of the immunogen, which is described generally in U.S. Pat. Nos. 5,484,735 and 4,629,692.

The immunogen can also be bound to an adjuvant or the adjuvant can be administered with the immunogen. Pharmaceutically acceptable adjuvants that can be useful in the context of the present invention to enhance the anti-heparanase immune response elicited by the immunogen, include, but are not limited to, muramyl peptides, lymphokines, such as interferon, interleukin-1 and interleukin-6, saponins and CpG oligonucleotides. The adjuvant can also be suitable particles onto which the immunogen is adsorbed, such as aluminum oxide particles. Other examples of pharmaceutically acceptable adjuvants that can be useful in the context of the present invention to enhance the anti-heparanase immune response are bacterial adjuvants. An example of a bacterial adjuvant is BCG. When functioning as an APC (described above) recombinant BCG can additionally act as its own adjuvant. In this case, an additional adjuvant may not be needed, although one or more additional adjuvants can optionally be present. When used in its natural state, BCG acts solely as an adjuvant by being combined with the immunogen, resulting in a form that induces an effective immune response.

In any of the embodiments of the above-described methods of eliciting an immune response against heparanase in an animal, the induced immune response against heparanase can induce production in the animal of either an antibody that specifically binds heparanase, CD4+ T helper cells or cytotoxic lymphocytes against the beparanase. T helper cells are triggered by antibodies to seek and attack invading organisms. Cells called macrophages summon T-helper cells to the site of the infection and present a protruding antigen onto which the t-helper cell locks, thus recognizing the invading substance. The T4-helper cell then reproduces and secretes its potent lymphokine hormones that stimulate B-cell production of antibodies; signal natural killer or cytotoxic (cell-killing) T-cells; and summon more macrophanges to the site of the infection. Th1, which is an acquired immune response whose most prominent feature is high cytotoxic T lymphocyte activity relative to the amount of antibody production, is promoted by CD4+ Th1 T-helper cells. Th2, which is an acquired immune response whose most prominent feature is high antibody production relative to the amount of cytotoxic T lymphocyte activity, is promoted by CD4+ Th2 T-helper cells. CD4 is a 55-kD glycoproteins originally defined as differentiation antigens on T-lymphocytes, but also found on other cells including monocytes/macrophages. CD4 antigens are members of the immunoglobulin supergene family and are implicated as associative recognition elements in MHS class II-restricted immune responses. On T-lymphocytes they define the helper/inducer subset. CD4 receptors are present on CD4 cells (helper T cells), macrophages and DC, among others. Normally, CD4 acts as an accessory molecule, forming part of larger structures (such as the T-cell receptor) through which T cells and other cells signal each other. Cytotoxic lymphocytes are immunized T lymphocytes that can directly destroy appropriate target cells. These cytotoxic lymphocytes may be generated in vitro in mixed lymphocyte cultures, in vivo during a graft-versus-host reaction, or after immunization with an allograft, tumor cell, or virally transformed or chemically modified target cell. The lytic phenomenon is sometimes referred to as cell-mediated lympholysis. These cells are distinct from natural killer cells and from killer cells mediating antibody-dependent cell cytotoxicity.

The present inventive immunogen can be administered for prophylactic and/or therapeutic treatments of various conditions. Treatment, in the context of the present invention, is intended to encompass inhibiting, slowing, or reversing the progress of the underlying condition, ameliorating clinical symptoms of a condition or preventing the appearance of clinical symptoms of the condition. It should be appreciated that the methods and compositions of the present invention can be used to treat any condition associated with heparanase activity in the animal and, thus, can be useful in treating conditions generally associated with excess heparanase activity. For example, the methods of the present invention can be used to treat conditions relating to injury, inflammation, diabetes or autoimmunity. Furthermore, the methods of the present invention can be used to treat an angiogenic condition, such as atherosclerosis, arthritis, macular degeneration and psoriasis. The identification of those suitable animals, including mammals such as rabbits, rats, mice, or, preferably, humans, that have conditions for which treatment with an immunogen that elicits an immune response to heparanase is well within the ability and knowledge of one skilled in the art. For example, any of the methods described herein for determining heparanase activity can be useful to determine animals having conditions for which treatment according to the present inventive methods is suitable.

In prophylactic applications, compositions containing the present immunogens are administered to a patient not presently actively suffering from the condition, but rather, have not yet exhibited symptoms or are in a symptom-free period of the condition, in an amount sufficient to at least partially reduce the future effects of the condition. Such an amount is also defined to be an “effective amount.” In this use, the precise amounts again depend upon the patient's state of health and general level of immunity, as well as dosing schedules, which are described below.

In therapeutic applications, compositions are administered to a patient already suffering from the condition in an amount sufficient to cure or at least partially arrest the condition. An amount adequate to accomplish this is defined as an “effective amount.” Amounts effective for this use will depend upon the severity of the condition and the general state of the patient's own immune system. Dosing schedules will also vary with the disease state and status of the patient and will typically range from a single bolus dosage or continuous infusion to multiple administrations per day, for example, every 4-6 hours, or as indicated by the treating physician and the patient's condition.

In another embodiment, the methods of the present invention can be used to inhibit tumor growth or prevent metastasis of a tumor, which is the growth of secondary tumors at sites different from the primary tumor. Examples of suitable tumors that can be treated by the method of this invention include the following: brain tumors, such as astrocytoma, oligodendroglioma, ependymoma, medulloblastomas and PNET (Primitive Neural Ectodermal Tumor); pancreatic tumors, such as pancreatic ductal adenocarcinomas; lung tumors, such as small and large cell adenocarcinomas, squamous cell carcinoma and bronchoalveolarcarcinoma; colon tumors, such as epithelial adenocarcinoma and liver metastases of these tumors; liver tumors, such as hepatoma and cholangiocarcinoma; breast tumors, such as ductal and lobular adenocarcinoma; gynecologic tumors, such as squamous and adenocarcinoma of the uterine cervix and uterine and ovarian epithelial adenocarcinoma; prostate tumors, such as prostatic adenocarcinoma; bladder tumors, such as transitional, squamous cell carcinoma; tumors of the RES System, such as B and T cell lymphoma (nodular and diffuse), plasmacytoma and acute and chronic leukemia; skin tumors, such as malignant melanoma; and soft tissue tumors, such as soft tissue sarcoma and leiomyosarcoma.

It is understood that the immunogens of the present invention, where used in an animal for the purpose of prophylaxis or treatment, can be administered in the form of a composition additionally having a carrier. Therefore, the present invention includes compositions for inhibiting heparanase activity in an animal of an immunogen and a carrier. Any suitable immunogen, examples of which are described above, can be used in the context of the present inventive composition. Suitable carriers, which can, for example, be in the form of a capsule, sachet, paper or other container, include, for example, one or more of water, saline, phosphate buffered saline, dextrose, glycerol, ethanol and the like, as well as combinations thereof. Carriers can further have minor amounts of auxiliary substances, such as wetting or emulsifying agents, preservatives or buffers, which enhance the shelf life or effectiveness of the binding polypeptides. When the carrier serves as a diluent, it can be a solid, semi-solid, or liquid material, which acts as a vehicle, excipient or medium for the active ingredient. Such compositions of the present invention are prepared in a manner well known in the pharmaceutical art.

The compositions of this invention can be in a variety of forms. These include, for example, solid, semi-solid and liquid dosage forms, such as tablets, lozenges, sachets, cachets, elixirs, suspensions, aerosols (as a solid or in a liquid medium), ointments containing, for example, up to 10% by weight of the active compound, soft and hard gelatin capsules, suppositories, suspensions, pills, powders, liquid solutions, dispersions, lyopholyzed forms, liposomes, injectable and infusible solutions, and sterile packaged powders and as a topical patch. The preferred form depends on the intended therapeutic application and mode of administration. For the purpose of this invention, the immunogen can also be administered by various routes, for example, by the oral or rectal route, topically or parenterally, for example by injection or infusion (intravenous, intraperitoneal, subcutaneous, intramuscular, intradermal). The compositions of the invention can, as is well known in the art, be formulated so as to provide quick, sustained or delayed release of the active ingredient after administration to the mammal.

Accordingly, the present invention can be used in vivo and in vitro for investigative, diagnostic, prophylactic, or treatment methods, which are well known in the art. For example, the cDNA and polypeptide sequence of full-length mouse heparanase may enable large-scale production of the polypeptide, benefiting heparanase protein structure studies, as well as screening and validation of heparanase inhibitors involved in the development of novel anti-cancer and anti-inflammation drugs.

Of course, it is to be understood and expected that variations in the principles of the invention herein disclosed can be made by one skilled in the art and it is intended that such modifications are to be included within the scope of the present invention.

All references mentioned herein are incorporated in their entirety.

### EXAMPLES

The examples that follow further illustrate the invention, but should not be construed to limit the scope of the invention in any way. Detailed descriptions of conventional methods, such as those employed in the construction of vectors and plasmids, the insertion of DNA or RNA encoding polypeptides into such vectors and plasmids, the introduction of plasmids into host cells and the expression and determination thereof of DNA, RNA and proteins can be obtained from numerous publications, including Sambrook et al., Molecular Cloning: A Laboratory Manual, 2^{nd} ed., Cold Spring Harbor Laboratory Press (1989).

Heparanase

Full-length mouse heparanase polypeptide was expressed in a bacterial expression system by methods known in the art. Briefly, the murine heparanase gene was cloned from a mouse embryo cDNA library and the full-length gene was cloned into the plasmid pET28a with a poly-histidine encoding sequence at the amino-terminal. The polypeptide was expressed in E. coli, purified from inclusion bodies by preparative electrophoresis and buffer exchanged into PBS and tested for endotoxin. All polypeptide preparations used in the animal studies contained <1.25 EU/ml of endotoxin.

Dendritic Cells (DC)

DC were isolated from the bone marrow of C57BL/6 syngeneic mice using the following protocol. Bone marrow cells were harvested from tibia and femurs, which cells were subsequently depleted of existing T cells, B cells, macrophages and granulocytes by incubating with an antibody cocktail (anti-CD4 (clone GK1.5), anti-CD8 (2.43), anti-Ia (B21-2), anti-B220 (RA3-3A1/6.1) and anti-Gr-1 (RB6-8C5/1)) for 30 min at 4° C. and then with rabbit complement for additional 30 min at 37° C. The remaining cells were cultured in 10% fetal calf serum (FCS) Dulbecco's Modified Eagles Medium (DMEM) in the presence of GM-CSF (20 ng/ml) and IL-4 (50 ng/ml) at 37° C., 5% CO2, for 3 days. Non-adherent cells were discarded and the remaining cells were cultured for an additional 3 days. The resulting non-adherent cells were transferred to new plates with fresh media and cultured for another 3 days. These mature DC were harvested and cryopreserved for later use as APCs for immunization experiments. Morphology and phenotypic analysis for the DC was verified by flow cytometry. Typically, DC have extensive dendrites, form cell clumps and express high levels of MHC class II, co-stimulatory molecules, such as B7.1/CD80 and B7.2/CD86, yet are negative for other cell lineage markers, such as the monocyte/macrophage marker CD14.

For pulsing with heparanase antigen, DC were washed twice in serum-free AIMV media and incubated with heparanase protein (100 μg/ml) in AIMV for 6-10 hours. The cells were washed twice in AIMV before being used.

### Example 1

The present example demonstrates administration to an animal of an effective amount of an immunogen that elicits an immune response to heparanase. DC, which are an example of an APC, were pulsed with heparanase (DC-heparanase). C57BL/6 mice were administered the DC-heparanase intravenously (i.v.) at a concentration of 5×10⁴ cells per mouse. In control groups, mice were administered i.v. either alkaline phosphatase (AP) pulsed DC (DC-AP or DC-control) or PBS. There were a total of three administrations given at intervals of ten days. Seven days after the last immunization, sera from all three groups of mice were tested for anti-heparanase antibody by ELISA.

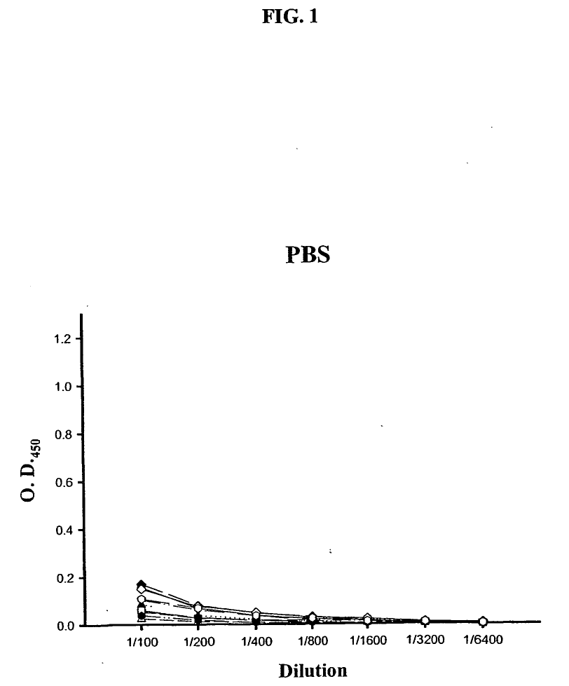

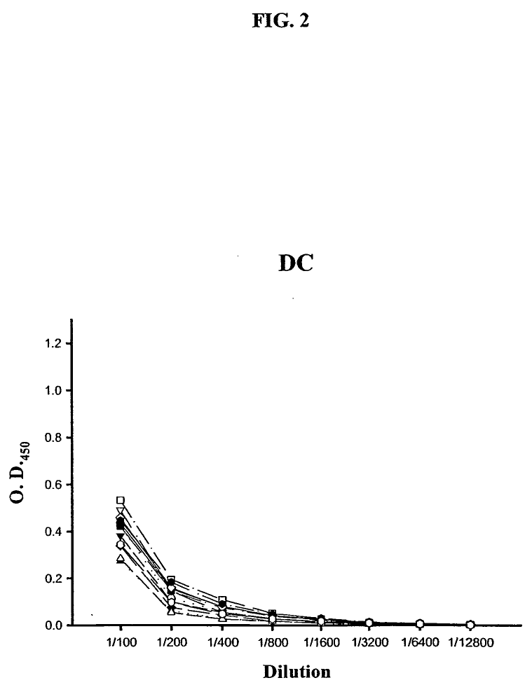

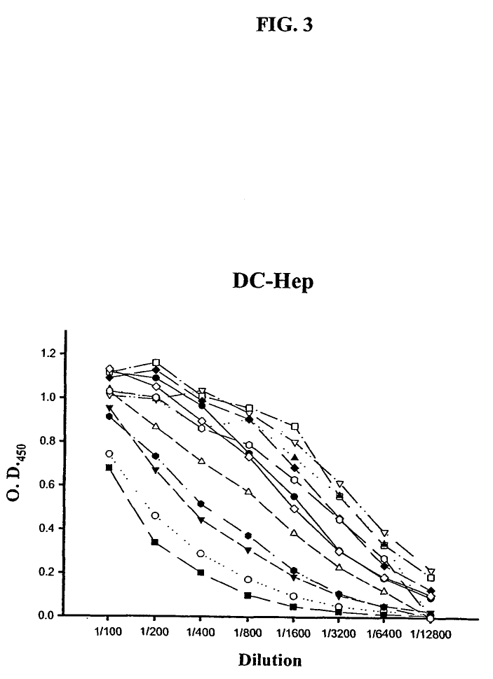

Results are shown in FIGS. 1-3, which are graphs of the concentration of antibody produced at various dilutions after administration of PBS (FIG. 1), DC-control (FIG. 2) and DC-heparanase (FIG. 3). These results indicate that a strong anti-heparanase antibody response was induced after administration of DC pulsed with heparanase, but not in the control groups. Anti-heparanase antibody was also not detectable in pre-immunization sera.

### Example 2

The present example demonstrates administration to an animal of an effective amount of an immunogen that elicits an immune response to heparanase. DC, which are an example of an APC, were pulsed with heparanase (DC-heparanase). C57BL/6 mice were administered the DC-heparanase i.v. at a concentration of 5×10⁴ cells per mouse. In control groups, mice were administered i.v. either chick ovalbumin (OVA) pulsed DC (DC-OVA or DC-control) or PBS. There were a total of two administrations given at intervals of 10 days. Seven days after the second administration, spleen cells were prepared and assayed using an Elispot assay. Briefly, spleen cells from the three experimental groups of mice were added at 2×10⁵ per well in 96-well flat-bottomed plates. Stimulated cells received 2×10⁴ DC-heparanase, DC-control, or PBS. Cells were cultured in RPMI 1640 with 10% fetal calf serum (FCS). After 4 days of in vitro culture, blue stained spots (due to antibody staining of IFN-γ released by single antigen-activated T cells) were counted and compared with plates set up with spleen cells from non-immunized or control-immunized mice.

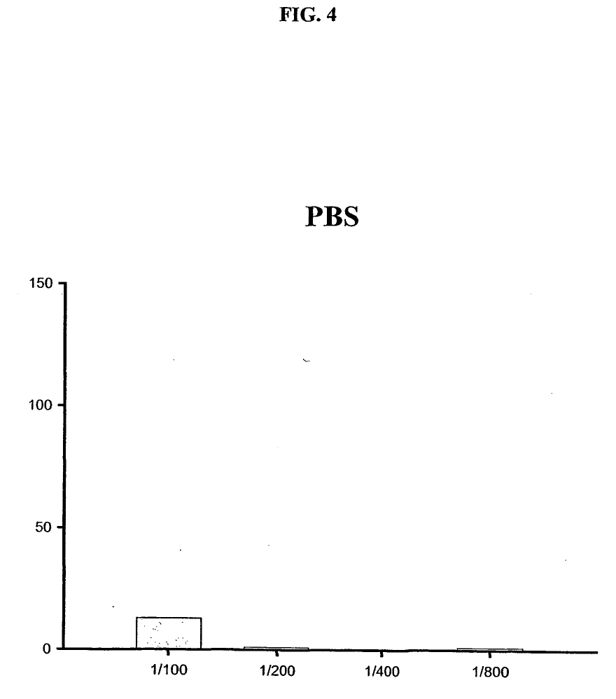

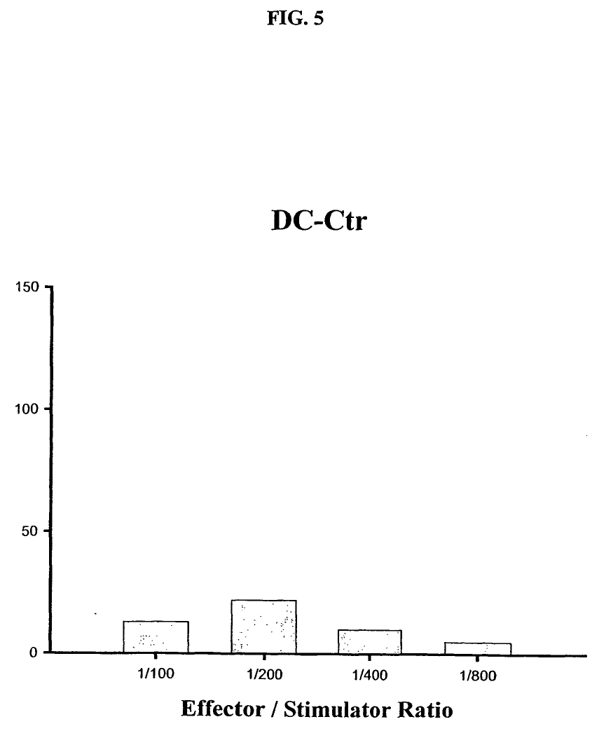

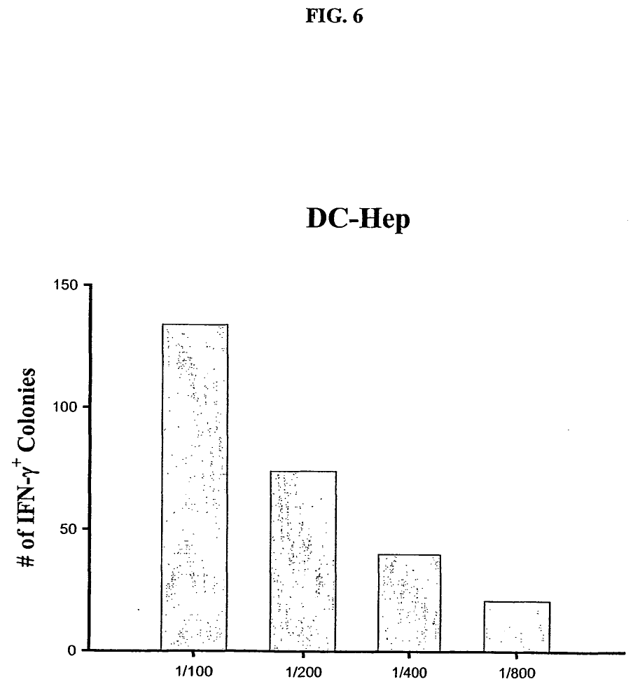

Results are shown in FIGS. 4-6, which are graphs of the number of spots producing INF-γ at different dilutions after administration of PBS (FIG. 4), DC-control (FIG. 5), or DC-heparanase (FIG. 6): These results indicate that there was a high frequency of heparanase-specific T cells in the mice immunized with DC pulsed with heparanase, but not in the control mice. In spleens from the mice that were administered DC-heparanase, the number of spots producing INF-γ is approximately ten times that in the control groups.

### Example 3

The present example demonstrates inhibition of tumor growth, including growth of tumor metastases, in an animal after administration to the animal of an effective amount of an immunogen. DC, which are an example of an APC, were pulsed with heparanase (DC-heparanase). C57BL/6 mice were administered the DC-heparanase i.v. at a concentration of 5×10⁴ cells per mouse. In control groups, mice were administered i.v. either DC pulsed with AP (DC-AP or DC-control) or PBS. There were a total of three administrations given at intervals of 10 days. Seven days later after the last immunization, mice were challenged by injecting 1×10⁶ Lewis lung carcinoma cells (a syngeneic tumor line) intrafootpad. When the tumor grew to approximately 5 mm in diameter, the tumor bearing leg was surgically removed. Mice were monitored daily for survival.

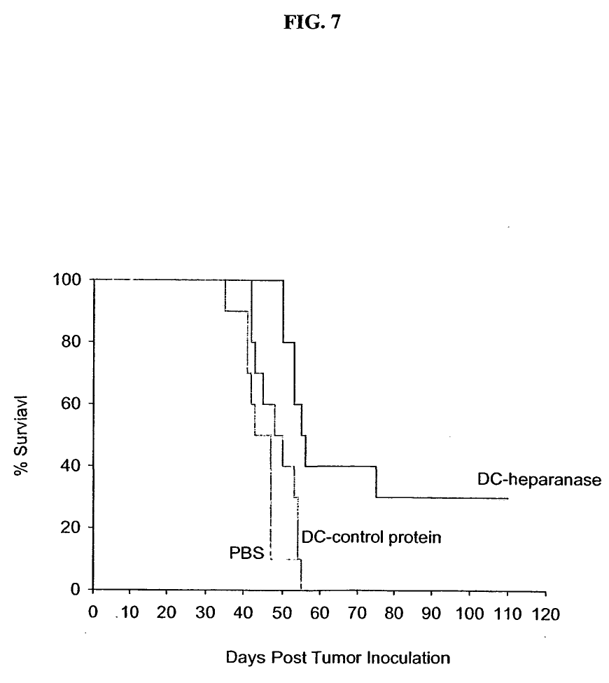

Results are shown in FIG. 7, which is a graph of percent survival as a function of time after administration of PBS, DC-AP (DC-control) and DC-heparanase. These results indicate that mouse survival was significantly prolonged in the DC-heparanase group, while all mice in the control groups died of lung metastases within twenty days after primary tumor removal. Thirty percent of DC-heparanase-immunized mice were considered cured because they were still alive more than 120 days post tumor challenged. These results indicate that immunization with DC-heparanase prolongs survival of tumor bearing mice in a metastasis model.

### Example 4

The present example demonstrates inhibition of tumor growth, including growth of tumor metastases, in an animal after administration to the animal of an effective amount of an immunogen. DC, which are an example of an APC, were pulsed with heparanase (DC-heparanase). C57BL/6 mice were administered the DC-heparanase i.v. at a concentration of 5×10⁴ cells per mouse. In control groups, mice were administered i.v. either DC pulsed with OVA (DC-OVA or DC-control) or PBS. There were a total of three administrations given at intervals of 10 days. Ten days later, mice were challenged by injecting 1×10⁶ B16 Melanoma cells. At day 60, mice were sacrificed, the lungs removed and tumor growth was measured by counting the number of tumor nodules on the lungs.

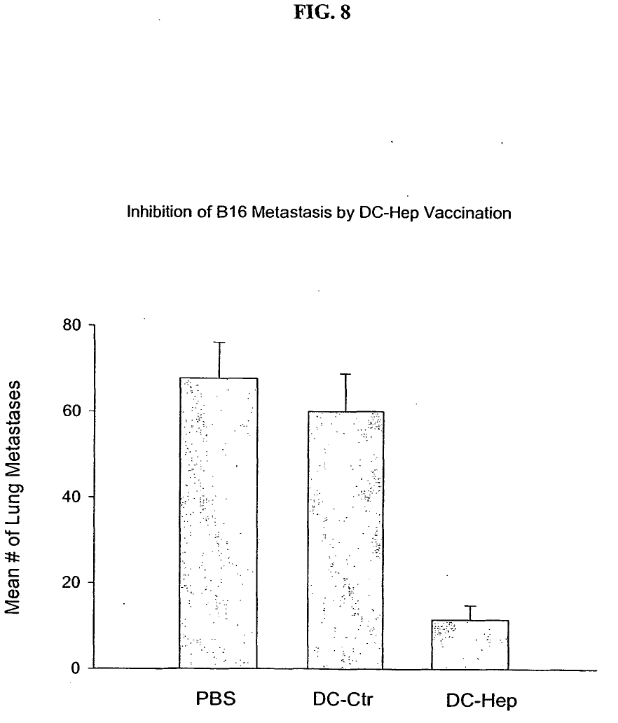

Results are shown in FIG. 8, which is a graph of the mean number of lung metastases after administration of PBS, DC-OVA (DC-control) and DC-heparanase. These results indicate that the mean number of lung metastases was significantly reduced in the DC-heparanase group. A strong inhibition of approximately 80% was found in the group of mice treated with DC-heparanase, as compared to only a slight inhibition of approximately 10% in the group of mice treated with DC-AP.

### Example 5

The present example demonstrates identification, expression, purification, and characterization of a polypeptide sequence of a mouse heparanase, which is set forth in SEQ ID NO:1, and a cDNA sequence of the mouse heparanase, which is set forth in SEQ ID NO:2.

Arrayed cDNA Library Screen

To obtain the full-length heparanase gene, a mouse (strain FVB) embryo (day 12.5) cDNA library was screened. A 96-well master plate of an oligo-dT primed library cloned into pCMV6-XL3 (Origene Technologies, Inc., Rockville, Md.) was screened by PCR. Two primers derived from the human gene sequence (forward 5′-CAAGAACAGC ACCTACTCAA GAAGC-3′, reverse 5′-GCCACATAAA GCCAGCTGCA AAGG-3′) were used for PCR screening. Positive subplate 10A was ordered and screened by PCR to identify positive subwells. The positive subwell stock was plated out onto LB-Ampicillin plates and colonies were screened to identify heparanase cDNA clones. A single clone with an insert of approximately 1800 bp was isolated and sequenced.

Expression

The open reading frame of the mouse gene was cloned into the plasmid pEE13.1 (Lonza Biologics, Inc., Fair Lawn, N.J.) by PCR using Vent polymerase (New England Biolabs, Beverly, Mass.). To establish a stable cell line, NS0 cells (Lonza Biologics) were transfected with linearized plasmid DNA by electroporation and cultured in glutamine-free DMEM with dialysed fetal calf serum and glutamine synthetase supplement (JRH Biosciences, Lenexa, Kans.). Several clones were screened for polypeptide expression by western blot using polyclonal anti-heparanase antibody and the highest producer was selected for culture in roller bottles.

Polypeptide Purification

The NS0 cells (8×10⁹) expressing mouse heparanase were harvested and treated with a buffer containing 1% Triton X-100, 500 mM NaCl, 15 mM sodium dimethyl glutarate, pH6.0 (30 min, 4° C.). After centrifugation, the extract was incubated with 20 ml Con-A beads (Amersham Pharmacia Biotech, Piscataway, N.J.) at 4° C., overnight, with gentle rocking. The bound material was eluted with 200 ml of 20% α-methyl mannoside, 500 mM NaCl, 15 mM sodium dimethyl glutarate, pH 6.0. The Con-A eluate was diluted with 1800 ml of 15 mM sodium dimethyl glutarate, pH 6.0, and loaded onto a Hi-Trap heparin-Sepharose column (Amersham Pharmacia). The column was eluted with a gradient of NaCl (0.025-1.5 M) in 15 mM sodium dimethly glutarate, pH 6.0, and all the fractions were tested for heparanase activity. The active fractions were pooled, concentrated with an Ultrafree concentrator (NMWL 30 K, Millipore, Bedford, Mass.), and subjected to size exclusion chromatography using a Superdex 75 column (Amersham Pharmacia). All the fractions were monitored by UV absorbance at 280 nm and tested for heparanase activity. Enzymatically active fractions were pooled for further characterization.

SDS-PAGE and Western Blot

Polypeptides were resolved by SDS-PAGE under reducing conditions using 4-20% gradient polyacrylamide gels. After electrophoresis, the gels were either stained with Coomassie blue or transferred to polyvinyldene difluoride membrane (Millipore). The membrane was probed with polyclonal antibody raised against human heparanase. After incubation with a goat anti-rabbit antibody conjugated to horseradish peroxidase, the blot was developed using a chemiluminescence substrate (Amersham Pharmacia).

Polypeptide Sequence Analysis and Mass Spectrometry

Polypeptides transferred onto polyvinyldene difluoride membrane were stained with Coomassie blue. Individual bands were cut out and polypeptide sequences were obtained by automated Edman degradation in an Applied Biosystems Procise Model 492 protein sequencer (Applied Biosystems, Norwalk, Conn.). Purifed polypeptide was directly analyzed by liquid chromatography-mass spectrometry (LC-MS) analysis using an Agilent 1100 HPLC with a Poros Reverse Phase R1/10 column (Agilent Technologies, Palo Alto, Calif.). The column was coupled to a Thermo-Finnigan LCQ Deca XP ion trap mass spectrometer.

Heparanase Activity

Polypeptide samples were mixed with 25 μl of ³⁵S-ECM-HSPG (a generous gift from Dr. Israel Vlodavsky, The Hadassah-Hebrew University Hospital, Jerusalem, Israel) in a buffer containing 0.1 M sodium acetate, pH 5.0, 0.01% Triton X-100. After overnight incubation at 37° C., the samples were loaded onto a Superose 12 column (0.7×25 cm, 0.5 ml/min) and the radioactivity of the fractions (0.25 ml/fraction) were measured in a β-scintillation counter. The enzymatic activity was also studied for the ability to degrade HS. For this purpose, ³H-HS-Sepharose (6.25 nM) was substituted as the substrate in the heparanase reaction, and the radioactivity released into the reaction buffer after overnight incubation was determined.

Cloning the Full Length Mouse Heparanase Gene

To isolate a full-length mouse heparanase gene, an arrayed mouse embryo cDNA library was screened using primers located in the middle of the heparanase coding sequence. Out of 5×10⁵ clones screened, a single positive clone containing an insert of approximately 1800 base pairs was isolated and sequenced. This clone contained an open reading frame of 1605 bp encoding a polypeptide of 535 amino acids with a theoretical molecular mass of 60064 Da. The clone included 81 base pairs upstream of the start codon. A consensus polyadenylation signal, AATAAA, was located downstream of the stop codon and 22 bp upstream of the poly(A) tail.

Polypeptide Expression, Purification and Characterization

The mouse gene was transfected into a mouse myeloma cell line (NS0) and stable clones were isolated using the glutamine synthetase selectable marker. Heparanase activity was detected in cell culture supernatants and detergent treated cell extracts, and expression was confirmed by western blot analysis. To purify the recombinant polypeptide, NS0 cells were treated with detergent and the extracts were incubated with Con-A sepharose beads overnight in a batch mode. The beads were washed and polypeptide was eluted with α-methyl mannoside.

Enzymatically active fragments were loaded onto a heparin-sepharose affinity column, the column was washed, and polypeptide was eluted using a salt gradient. Active fractions from this step were concentrated and the polypeptides were resolved by size exclusion chromatography. When the active fractions were run on a SDS-PAGE gel, the major bands detected were at 50 and 8 kDa, which is similar to the human polypeptide.

Western blot analysis of the purified recombinant mouse polypeptide also detected a small amount of a higher molecular weight species of 65-70 kDa that may represent full-length unprocessed polypeptide and is consistent with known human heparanase. The N-terminal sequence of the 50-kDa band was KEF XST YSR SSV DML YSF AKC SGL DLI FG, and DDV VDL EFY TKR PLR SVS PSF LSI TID ASL for the 8-kDa peptide as determined by Edman degradation. The fourth residue of the 50-kDa polypeptide was not detected during sequencing, suggesting this site is glycosylated. LC-MS analysis of the 8 kDa peptide revealed a molecular mass of 8051.0, which strongly suggests that the peptide corresponds to the 71 amino acid peptide Asp²⁹-Lys¹⁰⁰, the mass of which is 8050 Da.

Mature human heparanase polypeptide may exist as a heterodimer of 50 kDa and 8 kDa peptides. The data reported here for the mouse enzyme are consistent with this idea. The 535 amino acid mouse pre-proheparanase is first processed into a 60-kDa proheparanase polypeptide by cleavage of the signal peptide. Subsequently, an internal 49-residue peptide (Glu¹⁰¹-Gln¹⁴⁹) is removed proteolytically, resulting in the mature enzyme that exists as a non-covalently bound heterodimer of the 50 kDa polypeptide (Lys¹⁵⁰-Ile⁵³⁵) and the 8 kDa peptide (Asp²⁸-Lys¹⁰⁰).

Comparisoin of the Mouse and Human Heparanases

The mouse and human heparanases were determined to be 77% identical at the amino acid level. The mouse polypeptide is eight residues shorter than the human heparanase (535 vs 543) and this difference could be attributed to the smaller mouse signal sequence. The smaller 8-kDa fragment of mouse heparanase is 2 amino acids shorter than the human polypeptide, while the larger polypeptide is the same length, 385 amino acids. Mouse heparanase has two fewer potential N-linked glycosylation sites than the human, but the four present in the mouse are conserved in human heparanase. It has been observed that the larger subunit of the purified mouse polypeptide migrates faster on a SDS-PAGE gel than the human enzyme even though they contain the same number of amino acid residues. This is consistent with the observation that there are fewer glycosylation sites present in the mouse heparanase.

Mouse Heparanase Activity

To test whether the recombinant mouse heparanase is enzymatically active, purified polypeptide was incubated with metabolically radiolabeled HSPG. The reaction products were analyzed by gel filtration on a Superose 12 column. Undigested ³⁵S-HSPG in the ECM eluted in the early fractions (5-15), whereas the substrates incubated with mouse heparanase eluted as low-MW fragments in later fractions (16-35). Purified human platelet heparanase used as control gave rise to a similar activity profile.

It has been demonstrated that human heparanase enzymatic activity is pH dependent. A similar activity profile was observed when mouse heparanase was incubated with ³H-HS at various pH conditions. The enzyme was most active at pH 5.0 where 50 ng heparanase resulted in a maximal degradation of 6.25 nmole ³H-HS. Heparanase at pH 7.5 was still able to degrade HS, but almost 10-fold more enzyme was necessary to achieve the comparable activity. Little or no enzymatic activity was detected when the mouse heparanase was incubated at pH 4.0 or 8.0.

Laminarin sulfate, which has been shown to inhibit human heparanase activity in vitro and tumor metastasis in vivo, completely inhibited mouse heparanase activity at 1 μM, with an IC₅₀ of 50 nM (FIG. 6). The enzyme activity was also inhibited by heparin and other heparanase inhibitors.

1. A method of inhibiting heparanase activity in an animal comprising administering to the animal an effective amount of an immunogen that elicits an immune response to heparanase.

2. The method of claim 1, wherein the immunogen comprises heparanase or a fragment thereof.

3. The method of claim 2, wherein the heparanase or a fragment thereof is a mouse heparanase or a fragment thereof.

4. The method of claim 1, wherein the immunogen comprises an antigen presenting cell (APC) having heparanase or a fragment thereof displayed on the surface.

5. The method of claim 4, wherein the APC is a dendritic cell.

6. The method of claim 1, wherein the immunogen comprises a DNA or RNA encoding heparanase or a fragment thereof.

7. The method of claim 6, wherein the DNA or RNA is delivered to the animal via a cloning or expression vector.

8. The method of claim 6, wherein the DNA or RNA encodes a mouse heparanase or a fragment thereof.

9. The method of claim 1, wherein the method induces production of an antibody that specifically binds heparanase.

10. The method of claim 1, wherein the method induces production of CD4+T helper cells.

11. The method of claim 1, wherein the method induces production of cytotoxic lymphocytes specific for heparanase.

12. The method of claim 1, wherein the method is used to treat a condition associated with heparanase activity in the animal.

13. The method of claim 12, wherein the condition is related to inflammation or injury.

14. The method of claim 12, wherein the condition is related to diabetes or autoimmunity.

15. The method of claim 12, wherein the condition is an angiogenic condition selected from the group consisting of atherosclerosis, arthritis, macular degeneration and psoriasis.

16. The method of claim 1, wherein the method inhibits tumor growth.

17. The method of claim 1, wherein the method prevents metastasis of a tumor.

18. The method of claim 1, wherein the animal is a mammal.

19. The method of claim 18, wherein the animal is a human.

20. An isolated heparanase polypeptide comprising SEQ ID NO:1 or a fragment thereof.

21. An isolated heparanase polynucleotide encoding a heparanase polypeptide comprising SEQ ID NO:2 or a fragment thereof.

22. A cloning or expression vector comprising the polynucleotide of claim 21.

23. A host cell comprising the cloning or expression vector of claim 22.

24. A composition for inhibiting heparanase activity in an animal comprising an immunogen and a carrier.

25. A composition for inhibiting heparanase activity in an animal comprising SEQ ID NO:1 and a carrier.

26. A composition for inhibiting heparanase activity in an animal comprising SEQ ID NO:2 and a carrier.


SEQUENCE LISTING (2 sequences)

SEQ ID NO: 1 - DNA, 1742 bp, Rodent
  CDS (88)...(1695)
gaattcggcacgagggttgcagggggcttgagccagcgcgccgggctgccccagctctcccggcagcgggcggtccagccaggtgggatgctgaggctgctgctgctgtggctctgggggccgctcggtgccctggcccagggcgcccccgcggggaccgcgccgaccgacgacgtggtagacttggagttttacaccaagcggccgctccgaagcgtgagtccctcgttcctgtccatcaccatcgacgccagcctggccaccgacccgcgcttcctcaccttcctgggctctccaaggctccgtgctctggctagaggcttatctcctgcatacttgagatttggcggcacaaagactgacttccttatttttgatccggacaaggaaccgacttccgaagaaagaagttactggaaatctcaagtcaaccatgatatttgcaggtctgagccggtctctgctgcggtgttgaggaaactccaggtggaatggcccttccaggagctgttgctgctccgagagcagtaccaaaaggagttcaagaacagcacctactcaagaagctcagtggacatgctctacagttttgccaagtgctcggggttagacctgatctttggtctaaatgcgttactacgaaccccagacttacggtggaacagctccaacgcccagcttctccttgactactgctcttccaagggttataacatctcctgggaactgggcaatgagcccaacagtttctggaagaaagctcacattctcatcgatgggttgcagttaggagaagactttgtggagttgcataaacttctacaaaggtcagctttccaaaatgcaaaactctatggtcctgacatcggtcagcctcgagggaagacagttaaactgctgaggagtttcctgaaggctggcggagaagtgatcgactctcttacatggcatcactattacttgaatggacgcatcgctaccaaagaagattttctgagctctgatgtgctggacacttttattctctctgtgcaaaaaattctgaaggtcactaaagagatcacacctggcaagaaggtctggttgggagagacgagctcagcttacggtggcggtgcacccttgctgtccaacacctttgcagctggctttatgtggctggataaattgggcctgtcagcccagatgggcatagaagtcgtgatgaggcaggtgttcttcggagcaggcaactaccacttagtggatgaaaactttgagcctttacctgattactggctctctcttctgttcaagaaactggtaggtcccagggtgttactgtcaagagtgaaaggcccagacaggagcaaactccgagtgtatctccactgcactaacgtctatcacccacgatatcaggaaggagatctaactctgtatgtcctgaacctccataatgtcaccaagcacttgaaggtaccgcctccgttgttcaggaaaccagtggatacgtaccttctgaagccttcggggccggatggattactttccaaatctgtccaactgaacggtcaaattctgaagatggtggatgagcagaccctgccagctttgacagaaaaacctctccccgcaggaagtgcactaagcctgcctgccttttcctatggtttttttgtcataagaaatgccaaaattgctgcttgtatatgaaaataaaaggcatacggtacccctgagacaaaaaaaaaaaaaaaaaa

SEQ ID NO: 2 - protein, 535 aa, Rodent
MLRLLLLWLWGPLGALAQGAPAGTAPTDDVVDLEFYTKRPLRSVSPSFLSITIDASLATDPRFLTFLGSPRLRALARGLSPAYLRFGGTKTDFLIFDPDKEPTSEERSYWKSQVNHDICRSEPVSAAVLRKLQVEWPFQELLLLREQYQKEFKNSTYSRSSVDMLYSFAKCSGLDLIFGLNALLRTPDLRWNSSNAQLLLDYCSSKGYNISWELGNEPNSFWKKAHILIDGLQLGEDFVELHKLLQRSAFQNAKLYGPDIGQPRGKTVKLLRSFLKAGGEVIDSLTWHHYYLNGRIATKEDFLSSDVLDTFILSVQKILKVTKEITPGKKVWLGETSSAYGGGAPLLSNTFAAGFMWLDKLGLSAQMGIEVVMRQVFFGAGNYHLVDENFEPLPDYWLSLLFKKLVGPRVLLSRVKGPDRSKLRVYLHCTNVYHPRYQEGDLTLYVLNLHNVTKHLKVPPPLFRKPVDTYLLKPSGPDGLLSKSVQLNGQILKMVDEQTLPALTEKPLPAGSALSLPAFSYGFFVIRNAKIAACI